(19) United States
(12) Patent Application Publication
(10) Pub. No.: US 2011/0295256 A1
(43) Pub. Date: Dec. 1, 2011

(54) DISTAL CLAVICLE PLATE WITH CORACOID-CLAVICULAR LIGAMENT RECONSTRUCTION

(76) Inventor: Anthony S. Unger, Bethesda, MD (US)

(21) Appl. No.: 13/088,415

(22) Filed: Apr. 17, 2011

Related U.S. Application Data
(60) Provisional application No. 61/348,228, filed on May 25, 2010.

(51) Int. Cl. A61B 17/80

(52) U.S. Cl. 606/71; 606/70

(57) ABSTRACT

Provided is a system for stabilizing a fractured clavicle, the system having a bone plate capable of being attached at the site of a distal clavicle fracture, the bone plate defining a ligament attachment through passage sized to permit passage there through of a coracoid ligament graft, the system further including a plate hole cap configured to securely engage into the upper portion of the through passage after the ligament graft has been secured to the clavicle.

### BACKGROUND OF THE INVENTION

1. Technical Field

The present invention relates to a device useful for orthopedic surgery and in particular to a bone plate implant for repair or reconstruction of a clavicle. More particularly, the present invention relates to a clavicle bone plate configured to support reconstruction of the coracoid-clavicular ligament.

2. Background Art

The clavicle is a small but long bone comprised primarily of cancellous and cortical tissue that forms the anterior portion of the shoulder girdle. It articulates at its medial end with the manubrium sterni and at its lateral end with the acromion process of the scapula. The clavicles serve to protect the brachial plexus and the medial nerve and blood vessels that are immediately internal to them. The clavicles further serve mechanically as a strut and the only skeletal connection between the arms and the torso.

Because of its key position in the shoulder girdle, a large portion of the surface of the clavicle is devoted to attachment sites for a significant number and variety of muscles and ligaments. Among these is the coracoid-clavicular ligament, which provides an important load transfer function holding the positional relationship of the clavicle to the scapula. This important ligament extends from the coracoid process of the scapula to an attachment point on the under surface of the clavicle. In the repair of a distal clavicle fracture it is very important that the site of the fracture be properly aligned and stabilized. Repair of a fractured clavicle is particularly troublesome because of the multidirectional forces imposed on the clavicle by its many attachments to different muscles and ligaments. The attachment of the clavicle to the scapula subjects the fractured site on the clavicle to significant torque resulting motion of the shoulder joint. Unlike a fracture to a limb, a major fracture of the clavicle is extremely difficult to set with an external splint or cast. For this reason, a variety of implantable bone plates, which are designed to be surgically attached to a fractured clavicle, have been developed over time.

Distal fractures of the clavicle are generally divided into three types. Type 1 fractures of the distal clavicle are non-displaced fractures and do not normally require surgery and are typically treated by application of an externally worn restraining brace to limit motion of the fracture site during the healing period. A Type 3 fracture is a comminuted fracture of the distal piece and only occasionally requires a surgical procedure to obtain a satisfactory result. Type 2 fractures of the distal clavicle, however, are those that have a fracture line between the coracoid-clavicular ligament connection site and the distal end of the clavicle. When a Type 2 fracture of the clavicle occurs the ligament is torn, which results in displacement of the fracture and, in turn, high rates of non-union and mal-union if the fracture is not adequately reduced. It is unfortunate that Type 2 fractures are the most commonly experienced fractures of the distal clavicle and almost always require an open surgical repair procedure to properly align and stabilize the site of the fracture.

While some progress in the alignment and fixation of clavicle fractures has been achieved using bone plates that bridge the site of the fracture, there has been little progress in the restoration of the lost function of important ligaments that results from such injuries. Conventionally, reconstruction of the coracoid-clavicular ligament is accomplished by surgically attaching a hamstring graft, either autologus or allograph, to the coracoid process of the scapula and up into an interference hole in the clavicle where it is fixed with an interference bio-absorbable screw. This attachment of the hamstring graft into an interference hole in the clavicle is complicated by the need to properly position a bone plate at the same site to obtain proper alignment and stabilization of the fracture site.

To date there has been no bone plate system provided that is configured to be capable of supporting repair of a fracture of the distal portion of the clavicle and also designed to facilitate reconstruction of the coracoid-clavicular ligament. What is needed is bone plate configured for attachment to the surface of the clavicle to stabilize and facilitate healing of a fracture while providing a coracoid ligament connection site located in the bone plate that is configured to securely receive a coracoid-clavicular ligament reconstruction.

### SUMMARY OF THE DISCLOSURE

It is a primary object of the present invention to meet each of the above identified needs by providing a bone plate capable of stabilizing a skeletal fracture site, the bone plate also having an element configured to receive and securely attach a portion of soft tissue of a patient.

It is another object of the invention to provide a bone plate capable of being secured to a fractured clavicle at the site of the fracture, the bone plate having an element configured to receive and secure a tissue graft for repair of a damaged ligament of the patient.

It is another object of the invention to provide a system for stabilizing a fractured clavicle, the system having a bone plate capable of being attached at the site of a distal clavicle fracture, the bone plate configured to have a concave contoured underside of the plate to approximately complement the natural contour of the upper surface of the clavicle of a patient, the bone plate defining a ligament attachment through passage sized to permit passage there through of a coracoid ligament graft, the system further including a plate hole cap configured to securely engage into the upper portion of the through passage after the ligament graft has been secured to the clavicle.

It is another object of the invention to provide a system for stabilizing a fractured clavicle, the fracture site on the clavicle being approximately coincident with the clavicular attachment site for the coracoid-clavivular ligament, the system including a contoured bone plate that defines a ligament attachment through passage, the bone plate being capable of secure attachment to the clavicle at the fracture site, the ligament attachment through passage of the bone plate being positioned in alignment with a site on the clavicle a tendon graft can be attached, a tendon attachment element for attaching a tendon graft to the clavicle, a plate hole cap configured to securely engage into the upper portion of the through passage after the ligament graft has been secured to the clavicle.

It is another object of the invention to provide a kit, the kit including at least one bone plate configured to be capable of securely stabilizing a fractured clavicle and providing an element configured to secure a tendon graft for repair of a damaged coracoid ligament.

It is another object of the invention to provide a method for repair of a bone fracture where the fracture site is approximately coincident to an attachment site of a ligament on the fractured bone.

### BRIEF DESCRIPTION OF THE DRAWINGS

The foregoing and other features of the disclosed device will become apparent to one skilled in the art to which the present invention relates upon consideration of the following description of exemplary embodiments with reference to the accompanying drawings, wherein:

FIGS. 1A-B respectively show a representation of the normal anatomy of the distal portion of the clavicle of the subject and a representation of a fracture of the distal portion of the clavicle and the associated damage to the coracoid-clavicular ligament.

FIGS. 2A-C respectively show a top, side, and end view of the bone plate of the present invention.

FIG. 3 shows an exploded view of the present invention, in association with the clavicle, the coracoid process of the scapula, and a tendon graft used for repair of a damaged coracoid-clavicular ligament.

FIG. 4 shows a representation of the present invention as used to repair a distal fracture of the clavicle and a repair of the coracoid-clavicular ligament.

### DETAILED DESCRIPTION OF PREFERRED EMBODIMENTS

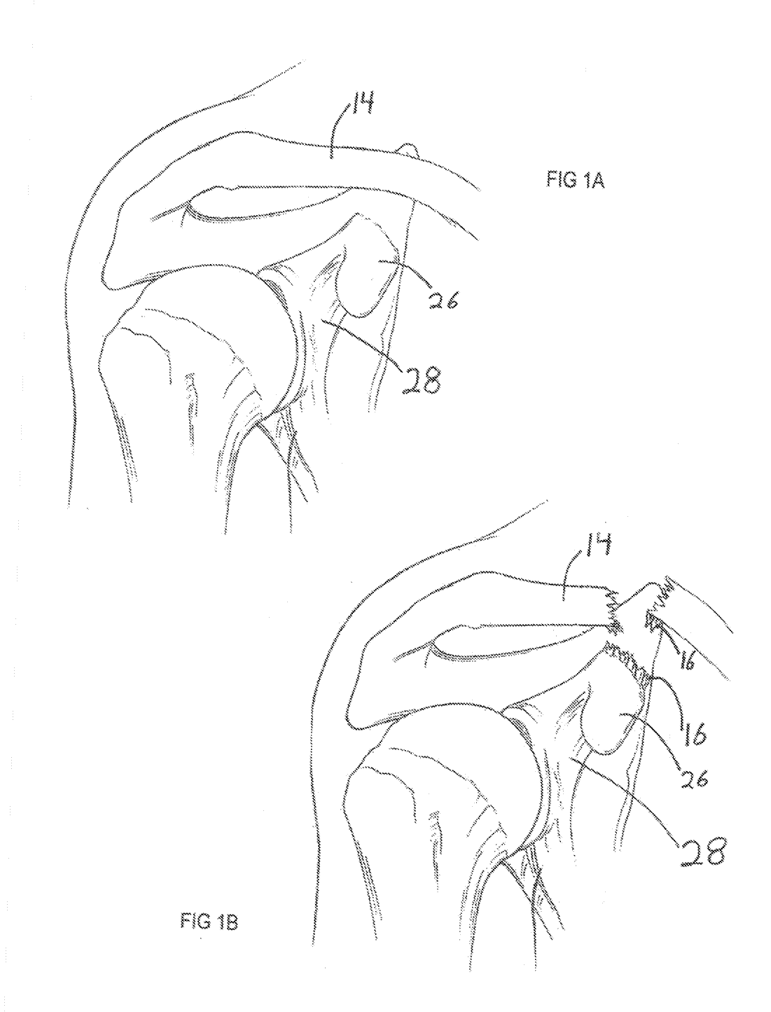

Detailed embodiments of the bone fracture repair system, generally shown at 10, and distal clavicle fracture bone plate 12 are disclosed herein and represented in FIGS. 1A-B, 2A-C, 3, 4, and 5; however, it is understood that the following description and each of the accompanying figures are provided as being exemplary of system and device. which may be embodied in various forms without departing from the scope of the claims. Thus, the specific structural and functional details provided in the following description are non-limiting, but serve merely as a basis for the system and device as defined by the claims provided herewith. The system described below can be modified as needed to conform to further development and improvement of materials without departing from the inventor's concept as defined by the claims.

The concept of the invention is to provide a system 10 and bone plate 12 for the fixation of a distal fracture of the clavicle 14 and the repair of a damaged coracoid-clavicular ligament 16. As presented in FIGS. 1A and 1B, a fracture of the distal portion of the clavicle can result in displacement of the clavicle and damage to the coracoid-clavicular ligament, which normally provides a load transfer function in stabilizing the scapula during normal motion of the shoulder joint. The present invention provides a system. 10 and method that permits stable fixation of the fracture site of the distal clavicle 14 by use of a hone plate 12 particularly configured to facilitate the restoration of the stabilizing function of the coracoid clavicular ligament 16 that is typically damaged by such clavicle fractures.

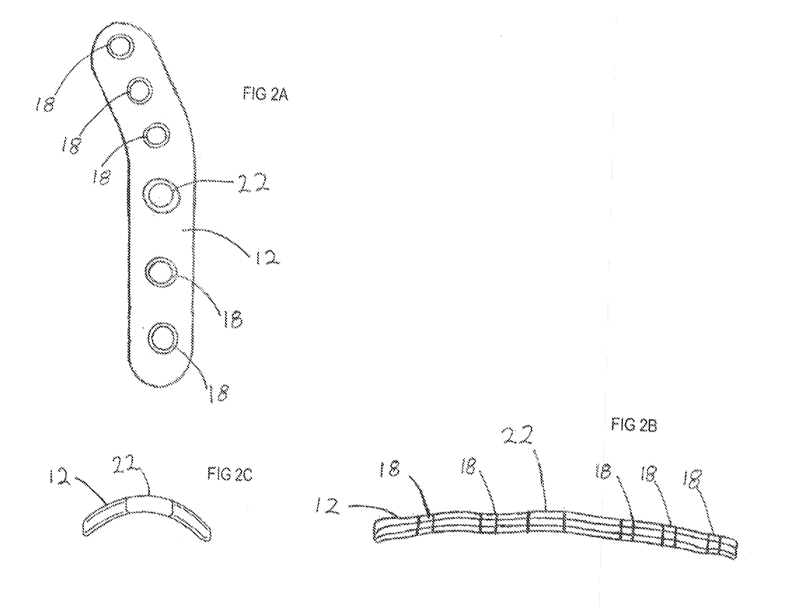

As shown in FIGS. 2A-C the bone plate 12 defines multiple screw holes 18, which are sized and configured to securely accept bone screws 20 capable of fixing the bone plate 12 flush against the surface of the fractured clavicle 14. Preferably, the bone plate 12 can be configured to have shapes, bends, and contours that conform to the natural shape of the bone. The screw holes 18 and associated bone screws 20 can be of different sizes as necessary.

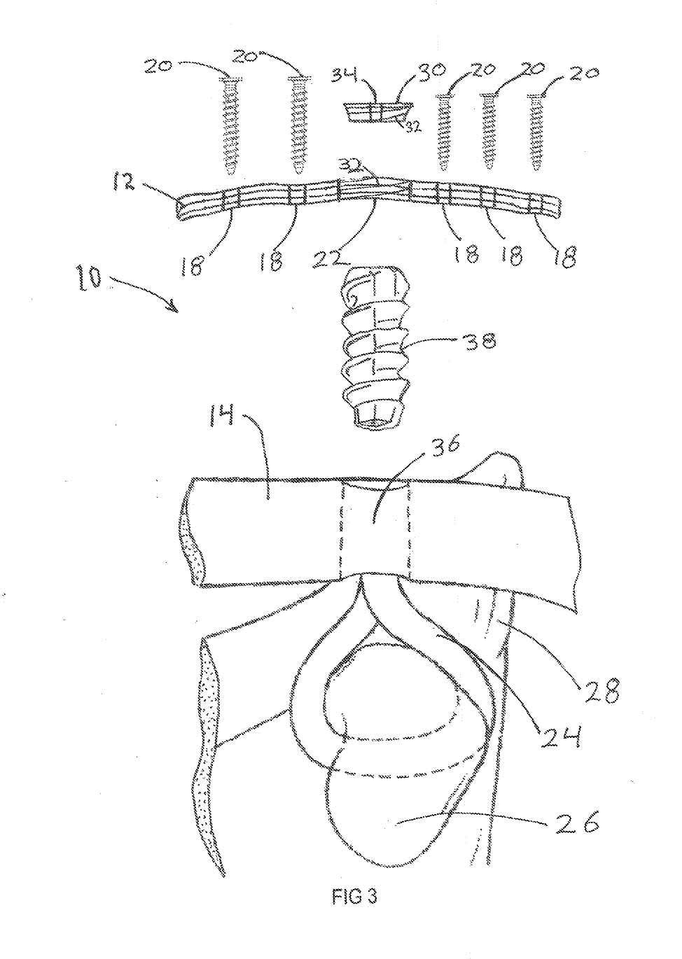

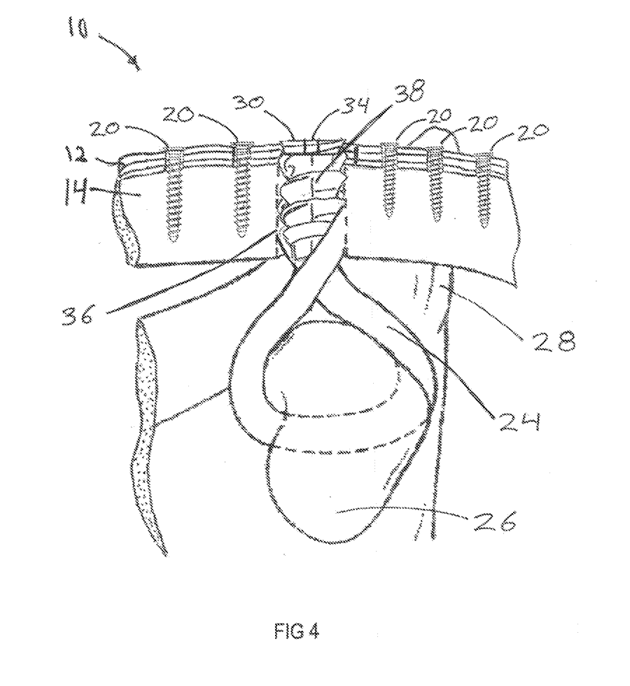

An important aspect of the bone plate 12 is a strategically positioned bone plate graft hole 22 that is defined through the plate 12 precisely at a position over the location on the clavicle 14 where a tendon graft 24 would best be passed into the clavicle 14 to allow anatomic reconstruction of the damaged coracoid-clavicular ligament 16. Unlike the screw holes 18 defined through the bone plate 12, the bone plate graft hole 22 would be required to have a dimension sufficient to allow passage of the graft 24 to be connected to the coracoid process 26 of the scapula 28. The bone plate graft hole 22 1.0 diameter size can be of any sufficient dimension to permit such ligament reconstruction, but preferably will, have a hole diameter of approximately 8 to 12 mm and more preferably will have a diameter of approximately 10 mm. Importantly, a graft hole cap 30 that is sized and configured to correspond to the size and configuration of the bone plate graft hole 22 defined in the bone plate 12 is also provided. The graft hole cap 30 can, upon completion of the ligament restoration procedure, be securely attached by the surgeon into the upper portion of the bone plate graft hole 22 of the bone plate 12. Preferably, the graft hole cap 30 and the bone plate graft hole 22 are provided with complementary threads 32; however, other secure attachment means, such as friction fit, snap fit, Luer lock attachment or any other means can be employed without departing from the concept of the invention. The graft hole cap 30 when so secured into the bone plate 12 serves to restore the strength and structural integrity of the bone plate 12. Alternatively, the graft hole cap 30 can be, as shown in FIGS. 3-4, provided with a bone screw passage hole 34 that, after being securely attached in the graft hole 22, permits passage of a bone screw 20 through the cap 30 and into the clavicle 14 beneath the bone plate 12. It is also possible that the bone screw 20 after partially passing through the bone screw passage hole 34 can contact, partially penetrate, and fix into the upper portion of the interference bioabsorbable screw 38.

FIG. 3 shows an exploded view of the components of the system 10 of the present invention in relation to a fractured clavicle 14 in need of fixation and the coracoid process 26 of the scapula 28 to which a tendon graft 24 can be attached to affect a repair of the coracoid-clavicular ligament 16.

The method of the present invention is best understood by reference to FIGS. 3-4, which depict the components of the system 10 and the present invention as used to repair a distal fracture of the clavicle 14 and a repair of the coracoid-clavicular ligament 16. As is known in the art, a surgeon would harvest a tendon graft 24 to provide a suitable replacement for the coracoid-clavicular ligament 16 damaged as a result of the distal fracture of the clavicle 14. Reconstruction of the coracoid-clavicular ligament 16 is commonly done using a hamstring graft 24 that can be either passed around the coracoid process 26 or, alternatively directly attached to the process 26 by fixation methods commonly known in the art. Exemplary of a method to effect attachment of the graft 24 to the coracoid process 26 is the Tightrope™ Graft Jacket™ system. Preliminary to attachment of the tendon graft 24 to the clavicle 14, the surgeon can drill a graft receiving hole 36 in the clavicle 14 for passage and reception of the tendon graft 24 into the clavicle 14. Preferably, the graft receiving hole 36 drilled in the clavicle 14 will have a diameter of 10 mm; however, a draft receiving hole 36 of any suitable dimension can be prepared in the clavicle 14 without departing from the concept of the method of the present invention. The surgeon, having looped or attached the tendon graft 24 to the coracoid process 26 of the scapula 28 and having prepared the graft receiving hole 36 in the clavicle 14, can then pass the graft 24 into and through the prepared graft receiving hole 36. The surgeon can then reduce the distal clavicle fracture and securely fix the fracture by positioning the bone plate 12 of the present invention across the site of the fracture using bone screws 20 as depicted in FIGS. 3-4. While the use of bone screws 20 is preferred, any other suitable means of attachment of the bone plate 12 to the clavicle 14 can be used without departing from the concept of the invention. Importantly, when fixing the bone plate 12 to the clavicle 14, the graft hole 22 defined in the bone plate 12 is positioned directly over the graft receiving hole 36 in the clavicle 14 through which the tendon graft 24 has been passed. The surgeon can then apply tension to the tendon graft 24 and fix the graft 24 to the clavicle 14 by mechanical insertion of an interference screw 38. Preferably, the commercially available bioabsorbable Tenodesis Screw™, available through Arthex, Inc., can be employed; however, any other suitable attachment means can be employed to secure the tendon graft 24 within the graft receiving hole 36 prepared in the clavicle 14. Once secured to the clavicle 14, the surgeon can cut off extraneous graft tissue that might extend through the bone plate graft hole 22. The plate hole cap 30 can then be secured into the upper portion of the bone plate graft hole 22 so as to restore the structural integrity and strength of the bone plate at this critical location.

The system 10 of the present invention, including the bone plate 12 provided with the specifically sized and configured bone plate graft hole 22, facilitates reconstruction of the coracoid-clavicular ligament 16 and secure fixation of the fracture site in the distal portion of the clavicle 14. While some components of the system of the present invention, such as the bone screws 20 employed to fix the bone plate 12 to the clavicle 14 and the interference bioabsorbable screw 38 can be obtained through commercially available sources, it is within the concept of the present invention that other bone plate attachment means and other non-proprietary graft attachment means can be suitably employed.

Materials, such as titanium, surgical steel, suitable alloys, and the like that are used in the construction of components of the system, such as the bone plate 12 and bone screws 20 are well known in the art.

While the preferred use of the present invention is described for the fixation of a fracture of the distal clavicle 14 with associated damage to the coracoid clavicular ligament 16, the principles of the system as disclosed herein can be adapted to provide for other skeletal fractures that occur in the body without departing from the concept of the invention.

It is also an advantage that the system can be provided as a kit having one or more bone plates 12 that can vary in size and configuration, as well as specific location of the bone plate graft hole 22 so as to permit the surgeon to select the proper components suitable for the particular anatomy of a patient.

The exemplary embodiment described above is provided for illustrative purposes only and it is within the concept of the present invention to include modifications and varying configurations without departing from the scope of the invention that is limited only by the claims included herewith.

What is claimed is:

1. A bone fracture fixation system, the system comprising:
a bone plate capable of being securely attached to an underlying bone, the bone plate, defining a bone plate graft hole;
a tendon graft attachment element, said tendon graft attachment element being sized and configured to permit passage of said tendon graft attachment element through said bone plate graft hole;
a graft hole cap, the graft hole cap being sized and configured to be capable of attachment within the upper portion of said bone plate graft hole.

2. The system of claim 1, wherein said bone plate is configured to be capable of attachment to a clavicle bone of a subject.

3. The system of claim 2, wherein said bone plate graft hole is sized and configured to receive a tendon graft for a damaged coracoid clavicular ligament of a subject.

4. The system of claim 3, wherein said tendon graft attachment element is an interference bioabsorbable screw that is sized and configured to secure a portion of said tendon graft within said bone plate graft hole.

5. The system of claim 1, wherein said graft hole cap is attachable within the bone plate graft hole by an attachment means selected from the group consisting of a screw attachment, friction fit attachment, luer lock attachment, and snap fit attachment.

6. The system of claim 1, wherein said graft hole cap defines a through passage configured as a bone screw passage hole.

7. The system of claim 1, wherein said bone plate defines at least one bone screw hole.

8. The system of claim 1, wherein said bone plate has an upper surface and a lower surface, said lower surface having a concave configuration to facilitate attachment of said lower surface against the natural curve of an underlying bone.

9. The system of claim 1, wherein said bone plate has an upper surface and a lower surface, said lower surface being configured to have varied shapes, bends, and contours that conform to the natural shape of the bone to provide a complementary contact of said lower surface to the contacting surface of the underlying bone.

10. A bone plate comprising:
a bone plate graft hole defined in said bone plate;
a graft hole cap, the graft hole cap being sized and configured to be capable of secure attachment within the upper portion of said bone plate graft hole.

11. The bone plate of claim 10, wherein said bone defines at least one bone screw hole.

12. The bone plate of claim 10, wherein said bone plate has an upper surface and a lower surface, said lower surface having a concave configuration to facilitate attachment of said lower surface against the natural curve of an underlying bone.

13. The bone plate of claim 10, wherein said bone plate has an upper surface and a lower surface, said lower surface being configured to have varied shapes, bends, and contours that conform to the natural shape of the bone to provide a complementary contact of said lower surface to the contacting surface of the underlying bone.

14. The bone plate of claim 10, wherein said graft hole cap is attachable within the bone plate graft hole by an attachment means selected from the group consisting of a screw attachment, friction fit attachment, luer lock attachment, and snap fit attachment.

15. The bone plate of claim 10, wherein said graft hole cap defines a through passage configured as a bone screw passage hole.

16. The bone plate of claim 10, wherein said bone plate graft hole is sized and configured to permit passage of a tendon graft attachment element through said bone plate graft hole.

17. A method of securely fixing the site of a fracture of the bone having associated damage to an attached ligament, the method comprising:
providing a tendon graft for repair of a damaged ligament connecting a first bone to a second bone;
attaching a first portion of said tendon graft to said first bone;
providing a bone plate, said bone plate comprising a bone plate graft hole defined through said bone plate;
reducing a bone fracture of said second bone, said bone fracture being at a location having associated damage to an attached ligament;
attaching said bone plate at the site of the bone fracture, said bone plate graft hole being positioned approximate to the damaged ligament;
repairing said damaged ligament;
providing a graft hole cap sized and configured to be capable of secure attachment within the upper portion of said bone plate graft hole;
securing said graft hole cap to said bone plate.

18. A kit for use in repairing a fractured bone, said kit comprising:
at least one bone plate, said bone plate comprising a bone plate graft hole defined in said bone plate, a graft hole cap, the graft hole cap being sized and configured to be capable of secure attachment within the upper portion of said bone plate graft hole;
a sterile container to contain said kit.

19. The kit of claim 18, wherein said kit comprises multiple bone plates having varied sizes and configurations suitable for selection to complement different sizes and configurations of a bone in need of fixation.

20. The kit of claim 19, further comprising at least one additional tool, instrument, or bone fixation device suitable for use in fixing a fractured bone.
